(19) United States
(12) Patent Application Publication
(10) Pub. No.: US 2023/0310531 A1
(43) Pub. Date: Oct. 5, 2023

(54) TRIPTERYGIUM WILFORDII COMPOSITION FOR INHIBITING DRUG-RESISTANT CANCER, AND PREPARATION METHOD AND USE THEREOF

(71) Applicant: HUAQIAO UNIVERSITY, Quanzhou City (CN)

(72) Inventors: Jinghong ZHANG, Quanzhou City (CN); Jingru OUYANG, Quanzhou City (CN); Xiuzhi HE, Quanzhou City (CN); Suxin YANG, Quanzhou City (CN)

(73) Assignee: HUAQIAO UNIVERSITY, Quanzhou City (CN)

(21) Appl. No.: 18/128,547

(22) Filed: Mar. 30, 2023

(30) Foreign Application Priority Data
Mar. 30, 2022 (CN) 202210329738.7

(51) Int. Cl. A61K 36/37; A61P 35/00

(52) U.S. Cl. CPC A61K 36/37; A61P 35/00

(57) ABSTRACT

The present disclosure provides a Tripterygium wilfordii composition for inhibiting a drug-resistant cancer. The composition is prepared by triptolide (TP) with any one or two compounds selected from the group consisting of 1β-(E-cinnamoyloxy)-4a-hydroxy-5a,7β,11-triacetoxy-8β-nicotinoyloxy-dihydroagarofuran, Demethylzeylasteral, Triptobenzene R, (3S,4S,5R,10S)-3,19-dihydroxy-7-ox-oabieta-8,11,13-triene, and Triptobenzene B. In the present disclosure, the composition has a lower toxicity and an enhanced drug effect for inhibiting proliferation of drug-resistant tumor cells than those of the TP. Compared with the single TP, the composition has an effect of attenuation and synergia.

### CROSS REFERENCE TO RELATED APPLICATION

This patent application claims priority to Chinese Patent Application No. 202210329738.7, filed with the China National Intellectual Property Administration on Mar. 30, 2022, the disclosure of which is incorporated by reference herein in its entirety as part of the present application.

### TECHNICAL FIELD

The present disclosure belongs to the technical field of biomedicine, and in particular relates to a Tripterygium wilfordii composition for inhibiting a drug-resistant cancer, and a preparation method and use thereof.

### BACKGROUND

Tripterygium wilfordii is a deciduous sprawling shrub belonging to the genus Tripterygium, family Celastraceae, whose roots, leaves, flowers, and fruits are used as medicines. Tripterygium wilfordii is mainly produced in Zhejiang, Anhui, Jiangxi, Hunan, Guangdong, Guangxi, Fujian, Taiwan, Yunnan and other places. Tripterygium wilfordii has an extremely complex set of active ingredients. The pharmacological effects of Tripterygium wilfordii mainly include immune regulation, anti-tumor, improvement of microcirculation, anti-inflammation, sterilization, antipyretic, and analgesic. Clinically, the Tripterygium wilfordii can be used to treat leprosy, rheumatoid arthritis, tuberculosis and other chronic diseases. At present, Tripterygium wilfordii tablets, Tripterygium glycoside tablets, and Tripterygium wilfordii syrup, granules, and admixtures have been produced, which can be used for the treatment of various diseases.

In recent years, the overall chemical composition of Tripterygium wilfordii, especially the evaluation of toxic effects of triptolides (TPs), has increasingly attracted the attention of scholars at home and abroad. However, due to the various components in the original plant, root, bark and leaves of the Tripterygium wilfordii, the content of many components in the original plant is extremely low. For example, the TP has a content of only 0.012% (120 mg/kg). This leads to the limitation of the application research of a single Tripterygium wilfordii composition. At present, more than 380 chemical components with a biological activity have been isolated and identified from the Tripterygium wilfordii. Some active molecules can inhibit tumors, such as the TP, but the TP has an extremely high pharmacological toxicity. For example, oral administration of TP can stimulate the gastric mucosa, causing nausea, vomiting, abdominal pain, diarrhea and other symptoms. The TP can also cause liver damages, skin pigmentation, rashes, and oral ulcers, and can lead to leukopenia. Therefore, it is a technical problem to be solved at this stage to develop a highly-efficient and low-toxic Tripterygium wilfordii composition.

### SUMMARY

In view of this, an objective of the present disclosure is to provide a Tripterygium wilfordii composition for inhibiting a drug-resistant cancer. The composition has an effect of inhibiting the drug-resistant cancer with high efficiency and low toxicity.

To achieve the above objective, the present disclosure provides the following technical solutions:

The present disclosure provides a Tripterygium wilfordii composition for inhibiting a drug-resistant cancer, including any two or more of the following compounds: A: 1-desacetylwilforgine; B: (2S)-5,7-dihydroxy-2′-methoxy-8-(3-methyl-2-butenyl)-4′,5′-furanone-flavanone (Tripteryol C); C: Triptergulide D; D: (3S,5R,6S,7E)-3,5,6-trihydroxy-7-megastigmen-9-one; E: 1β-(E-cinnamoyloxy)-4a-hydroxy-5a,7β,11-triacetoxy-8β-nicotinoyloxy-dihydroagarofuran; F: Tripterygiumine C; G: triptolide (TP); H: Demethylzeylasteral; I: Triptobenzene R; J: (3S,4S,5R,10S)-3,19-dihydroxy-7-ox-oabieta-8,11,13-triene; K: Tricosanoic acid; L: Wilfordiol B ((7R,8S)-8-O-4′-(3′,5′-dimethoxy-1′-hydroxymethylphenyl)-guaiacylglycerol); M: (+)-(7R,8S,8'S)-9-benzoyloxy-5-methoxy-lariciresinol; N: Triptersinine O (1β,5a-difuroyloxy-4a-hydroxy-7β,11-diacetoxy-8β-nicotinoyloxy-dihydroagarofuran); O: Triptregeline J (2β-acetoxy-1a,9β-dibenzoyloxy-4β-hydroxy-6β-(3-nicotinoyloxy)-3-dihydroagarofuran); P: Triptobenzene B; Q: Triptersinine E (1β-Z-cinnamoyloxy-4a-hydroxy-5a,7β,11-triacetoxy-8β-nicotinoyloxy-dihydroagarofuran); and R: Triptersinine Z3 (5α,11-diacetoxy-1β-trans-cinnamoyl-4α,7β,8β-trihydroxy-dihydroagarofuran).

Preferably, the composition is a combination of the G and one or more selected from the group consisting of the E, the H, the I, the J, and the P.

Preferably, when the composition is a combination of the G and one compound of the E, the H, the I, the J, and the P, the G and the one compound are at a volume ratio of 1:4 to 4:3.

Preferably, when the composition is a combination of the G and two compounds of the E, the H, the I, the J, and the P, the G and the two compounds are at a volume ratio of 1:1:1 to 4:3:4.

Preferably, the G, the E, the I, and the J each have an effective concentration of 0.5 μg/ml to 7 μg/ml.

Preferably, the H and the P each have an effective concentration of 10 μg/ml to 45 μg/ml.

The present disclosure further provides a preparation method of the Tripterygium wilfordii composition, including the following steps: subjecting compounds in a mixture of a Tripterygium wilfordii suspension cell extracting solution-derived analytical sample and a Tripterygium wilfordii suspension cell culture solution-derived analytical sample to high-performance liquid chromatography (HPLC) separation, and combining obtained separated compounds.

Preferably, the HPLC separation is conducted at the following conditions: a chromatographic column of Hypersil ODS2-C18 250 mm, a column temperature of a room temperature, absorption wavelengths of 210 nm, 219 nm, and 230 nm, a sample injection volume of 20 μl, a mobile phase of acetonitrile and ultrapure water, and elution conditions as follows:

| Time | Acetonitrile | Ultrapure | Flow rate |
|---|---|---|---|
| (min) | (%) | water (%) | (mL/min) |
| 0 | 10 | 90 | 0.5 |
| 10 | 16 | 84 | 0.5 |
| 35 | 30 | 70 | 0.5 |
| 36 | 30 | 70 | 1 |
| 50 | 30 | 70 | 1 |
| 80 | 100 | 0 | 0.5 |
| 90 | 10 | 90 | 0.5 |

The present disclosure further provides use of the composition in preparation of a drug for inhibiting a drug-resistant cancer.

Preferably, the cancer includes any one or more of lung cancer, gastric cancer, pancreatic cancer, breast cancer, and leukemia.

Compared with the prior art, the present disclosure has the following beneficial effects:

In the present disclosure, 18 kinds of active components are separated by HPLC using a separation solution and a culture solution of Tripterygium wilfordii cells obtained by culturing the Tripterygium wilfordii cells. The separation has a simple procedure, easy operation, and high reproducibility.

In the present disclosure, compared with TP, the composition of five new compounds obtained by screening for the first time has a reduced toxicity and an enhanced drug effect for inhibiting proliferation of drug-resistant tumor cells than those of the TP. Compared with the single TP, the composition has a substantial progress in attenuation and synergia.

### BRIEF DESCRIPTION OF THE DRAWINGS

FIGS. 1A-R show a drug-inhibitory effect of proliferation of 18 kinds of compounds on H460/DDP cells after 48 h of action;

FIGS. 2A-J shows a drug-inhibitory effect of proliferation of 5 kinds of compounds on A549 and H1299 cells after 48 h of action (where FIG. 2A to FIG. 2E are for A549/DDP cells; and FIG. 2F to FIG. 2J are for H1299/DDP cells);

FIGS. 3A-E show effects of TP, preferred compounds, and combinations thereof on A549/DDP cells after 48 h of inhibiting cell proliferation;

FIGS. 4A-E show a dose-effect curve after TP, active compounds, and combinations thereof are administered to the A549/DDP cells for 48 h;

FIGS. 5A-E show a proliferation inhibitory effect of TP, active molecular compositions, and combinations thereof on the H1299/DDP cells after 48 h of action;

FIGS. 6A-E show a dose-effect curve after TP, active compounds, and combinations thereof are administered to the H1299/DDP cells for 48 h;

FIGS. 7A-E show an effect of TP, active molecules, and combinations thereof on H460/DDP cells after 48 h of inhibiting cell proliferation;

FIGS. 8A-E show a dose-effect curve of TP, active molecules, and combinations thereof acting on the H460/DDP cells for 48 h; and

FIGS. 9A-E show a cytotoxic effect of TP, active molecules, and combinations thereof on 16HBE cells for 48 h.

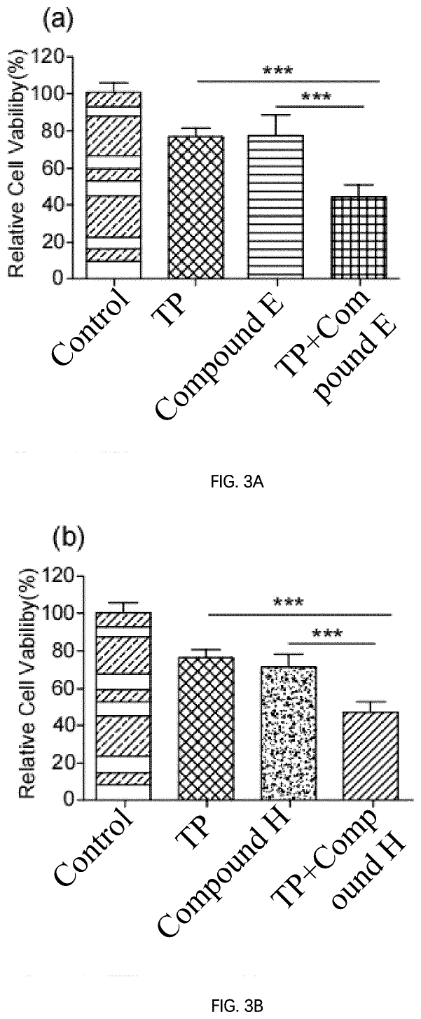

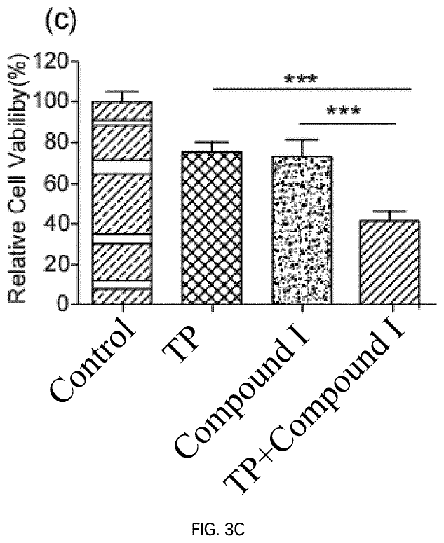

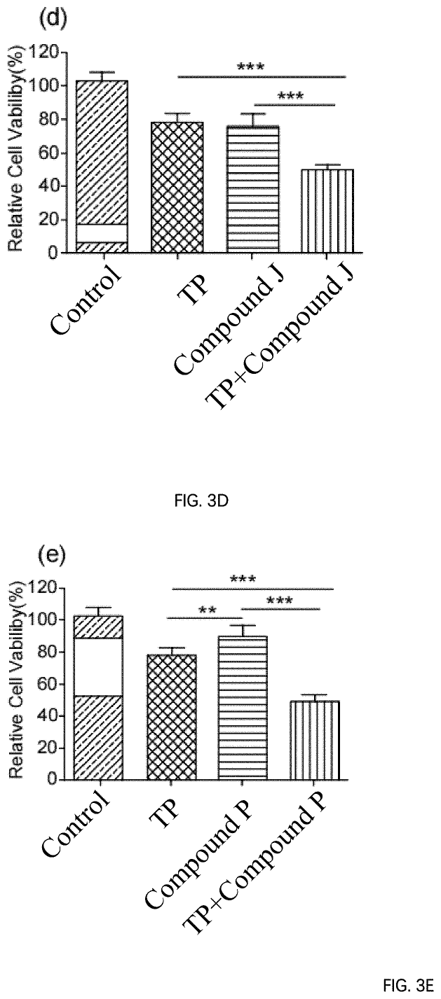

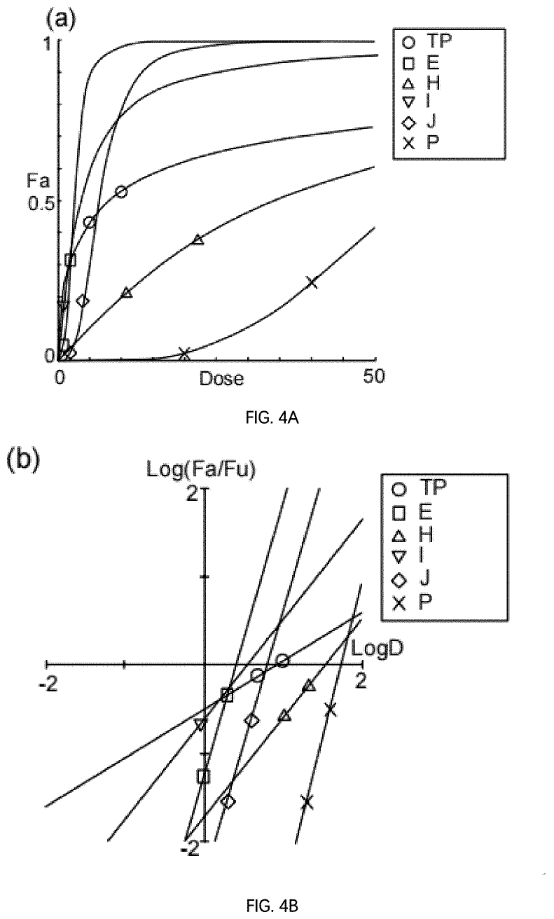

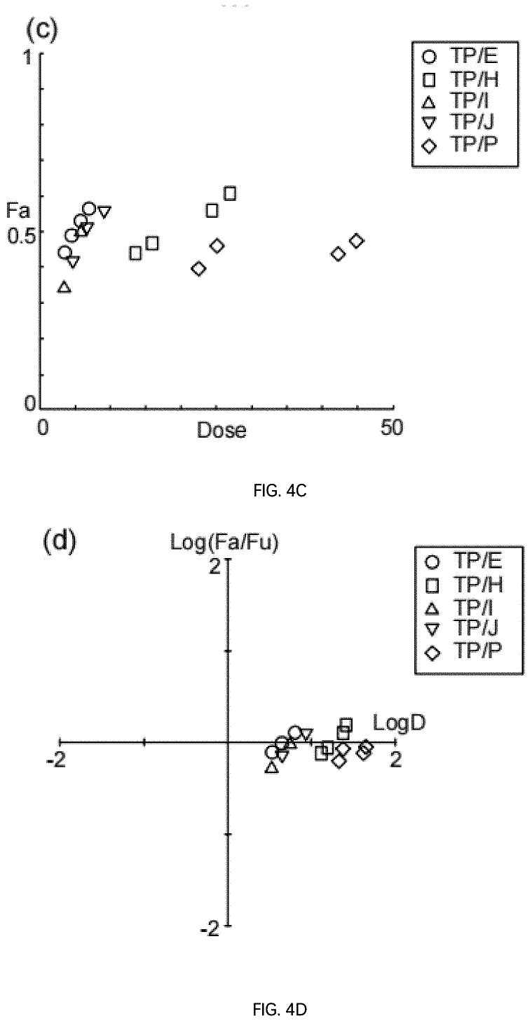

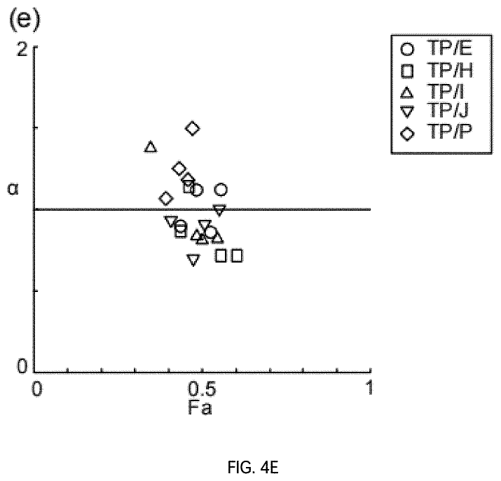

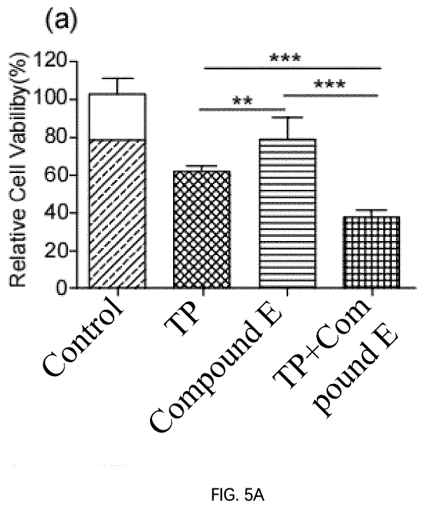

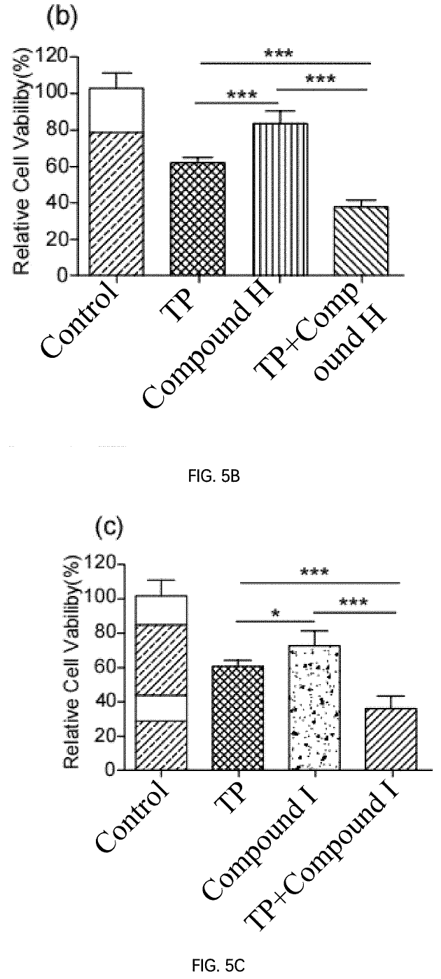

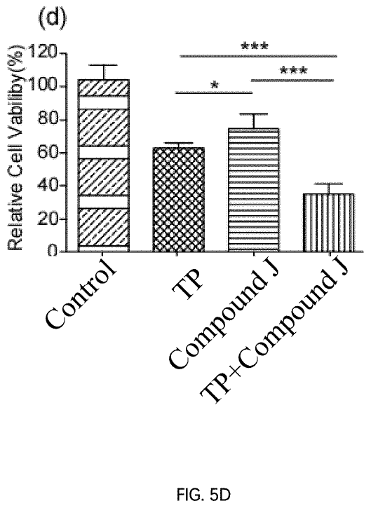

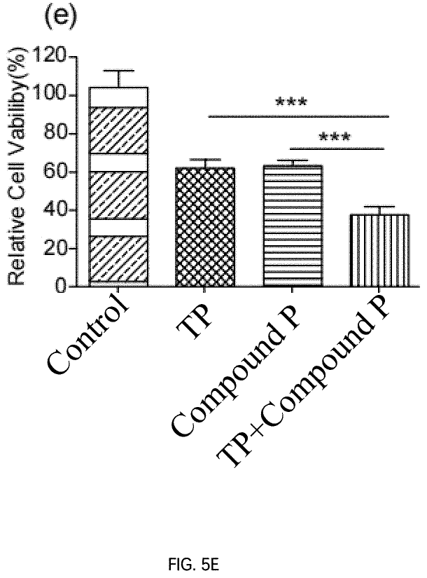

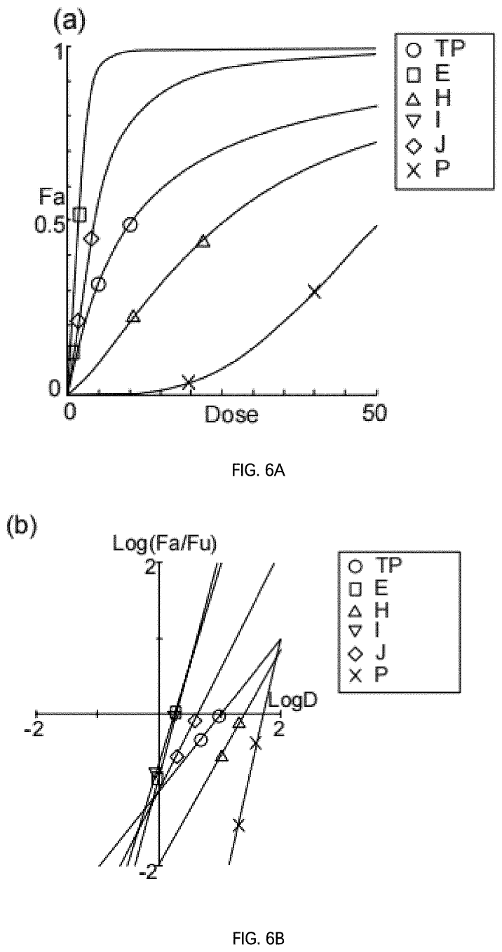

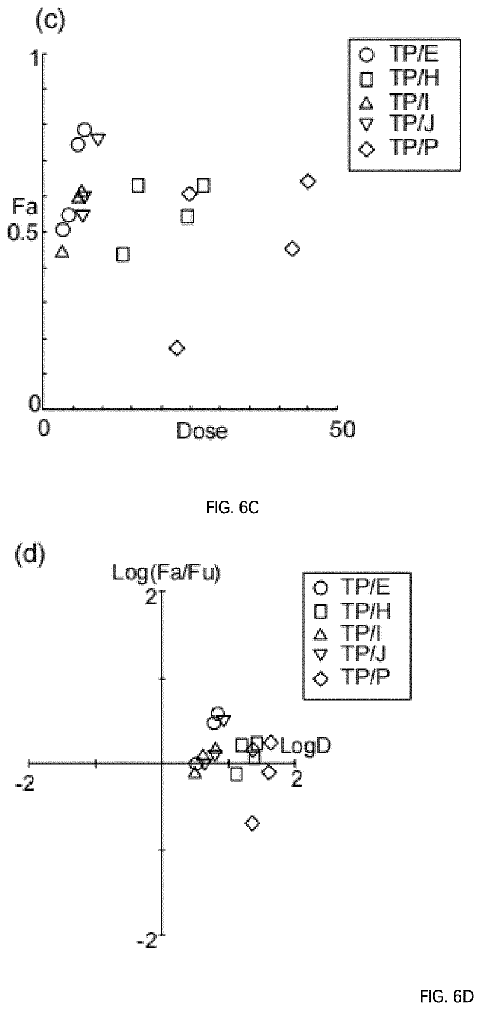

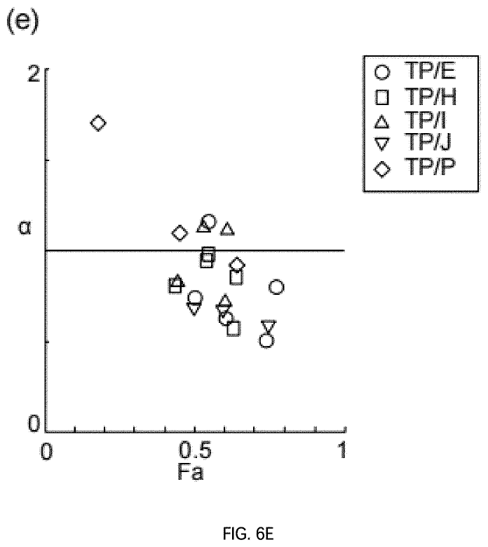

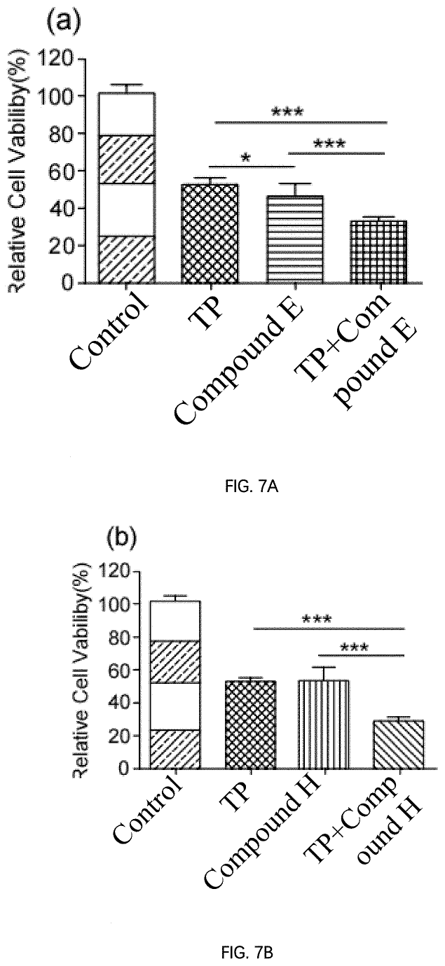

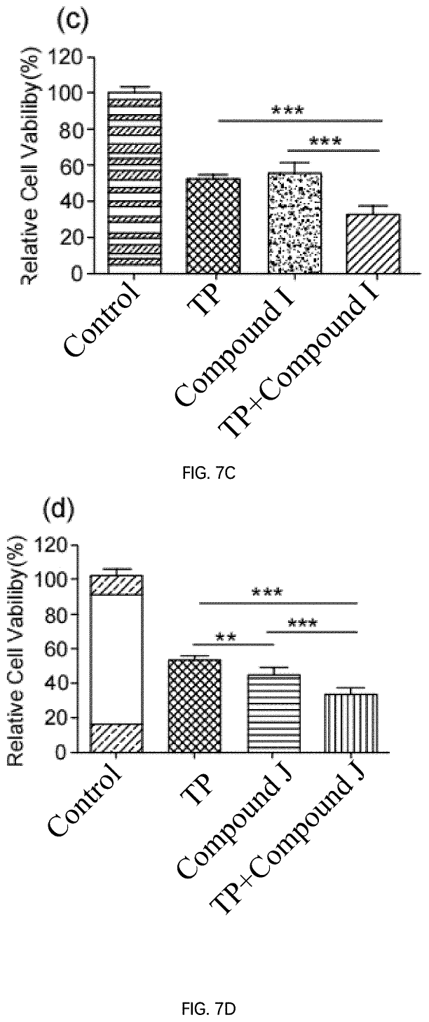

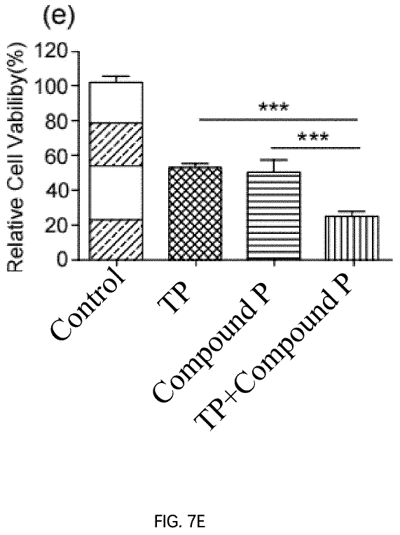

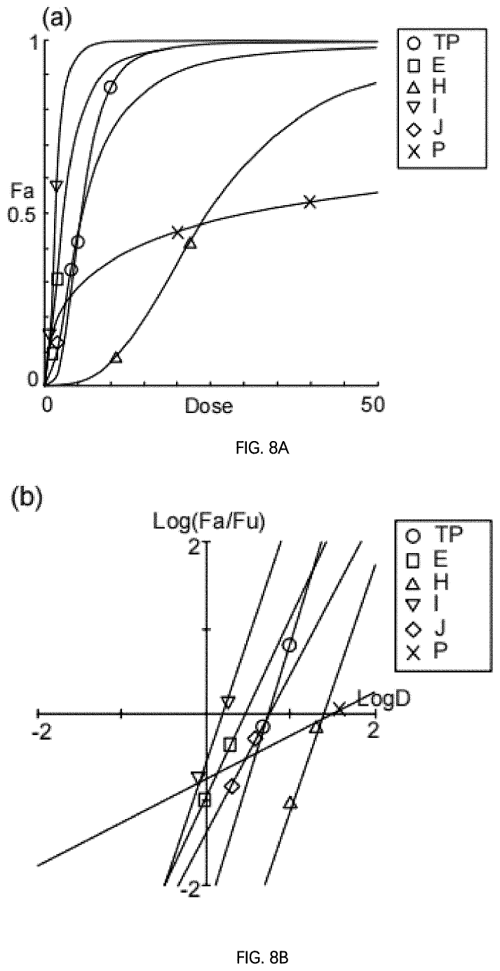

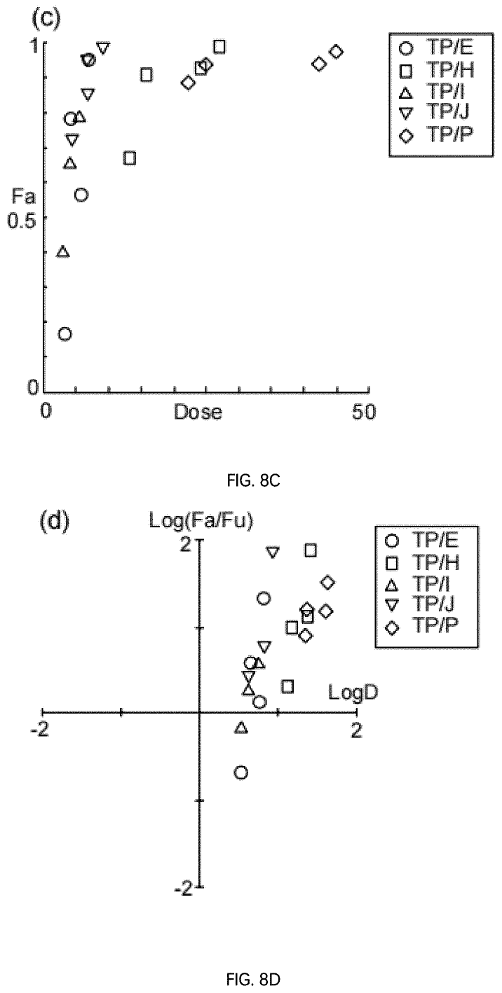

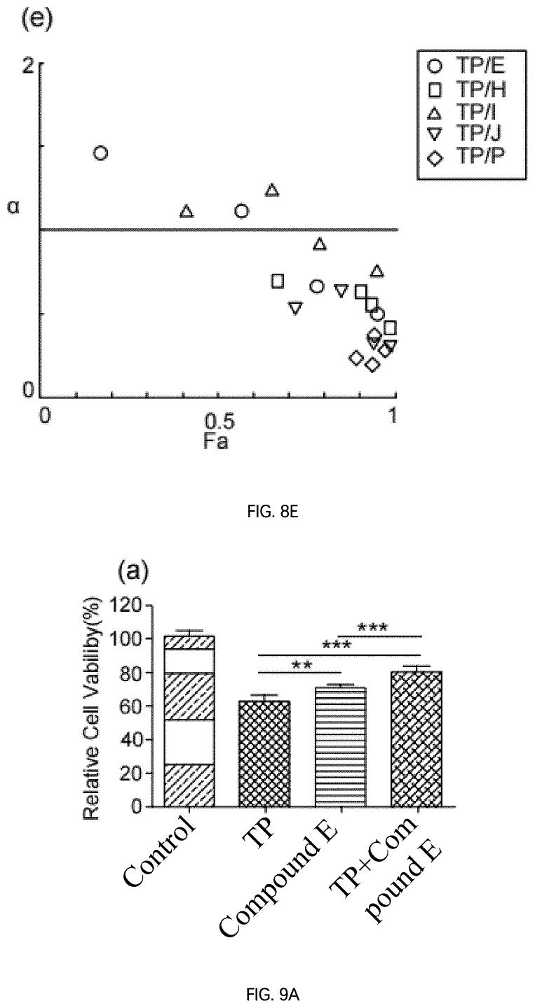

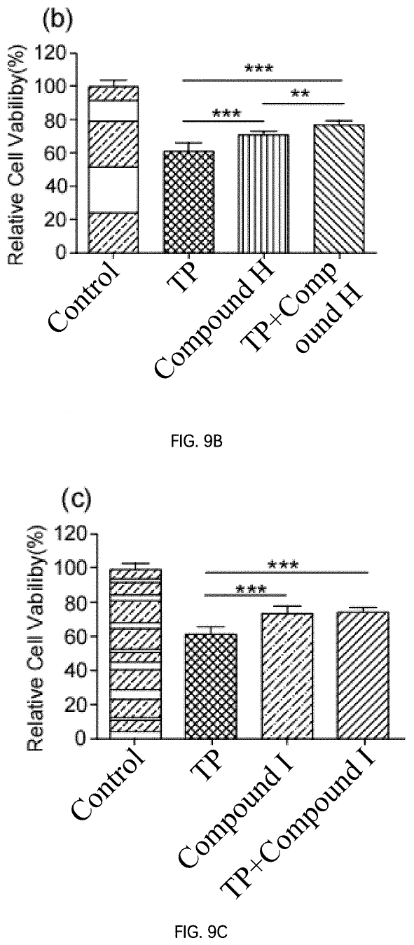

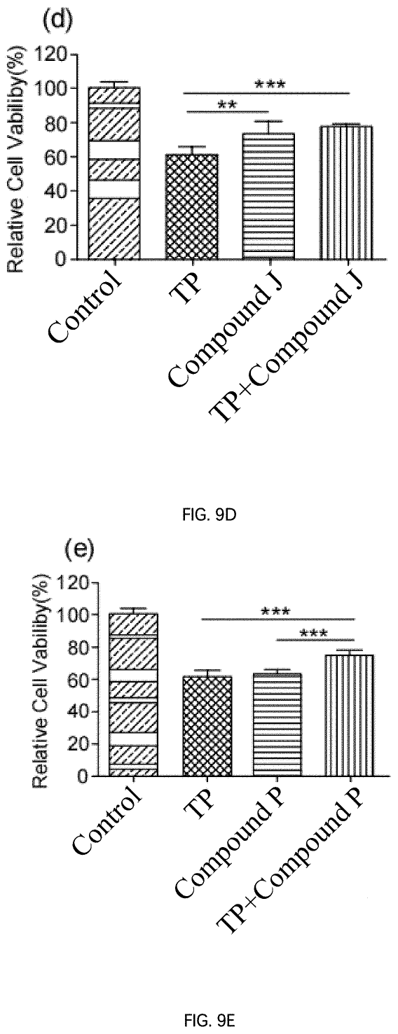

Notes: In FIGS. 3A-E, FIGS. 5A-E, FIGS. 7A-E, and FIGS. 9A-E, a is the compound E, b is the compound H, c is the compound I, d is the compound J, and e is the compound P. In FIGS. 4A-E, FIGS. 6A-E, and FIGS. 8A-E, a is a dose-effect curve of TP and active substances, b is a median effect of the TP and active substances, c is a dose effect of a combination of the TP and active substances, d is a median effect of the combination of the TP and active substances, and e is an Fa-CI diagram of the TP and active substances.

### DETAILED DESCRIPTION OF THE EMBODIMENTS

The present disclosure provides a Tripterygium wilfordii composition for inhibiting a drug-resistant cancer, including any two or more of the following compounds: A: 1-desacetylwilforgine; B: (2S)-5,7-dihydroxy-2′-methoxy-8-(3-methyl-2-butenyl)-4′,5′-furanone-flavanone (Tripteryol C); C: Triptergulide D; D: (3S,5R,6S,7E)-3,5,6-trihydroxy-7-megastigmen-9-one; E: 1β-(E-cinnamoyloxy)-4a-hydroxy-5a,7β,11-triacetoxy-8β-nicotinoyloxy-dihydroagarofuran; F: Tripterygiumine C; G: triptolide (TP); H: Demethylzeylasteral; I: Triptobenzene R; J: (3S,4S,5R,10S)-3,19-dihydroxy-7-ox-oabieta-8,11,13-triene; K: Tricosanoic acid; L: Wilfordiol B ((7R,8S)-8-O-4′-(3′,5′-dimethoxy-1′-hydroxymethylphenyl)-guaiacylglycerol); M: (+)-(7R,8S,8'S)-9-benzoyloxy-5-methoxy-lariciresinol; N: Triptersinine O (1β,5a-difuroyloxy-4a-hydroxy-7β,11-diacetoxy-8β-nicotinoyloxy-dihydroagarofuran); O: Triptregeline J (2β-acetoxy-1a,9β-dibenzoyloxy-4β-hydroxy-6β-(3-nicotinoyloxy)-β-dihydroagarofuran); P: Triptobenzene B; Q: Triptersinine E (1β-Z-cinnamoyloxy-4a-hydroxy-5a,7β,11-triacetoxy-8β-nicotinoyloxy-dihydroagarofuran); and R: Triptersinine Z3 (5α,11-diacetoxy-1β-trans-cinnamoyl-4α,7β,8β-trihydroxy-dihydroagarofuran).

In the present disclosure, the structural formulas of each compound are as follows:

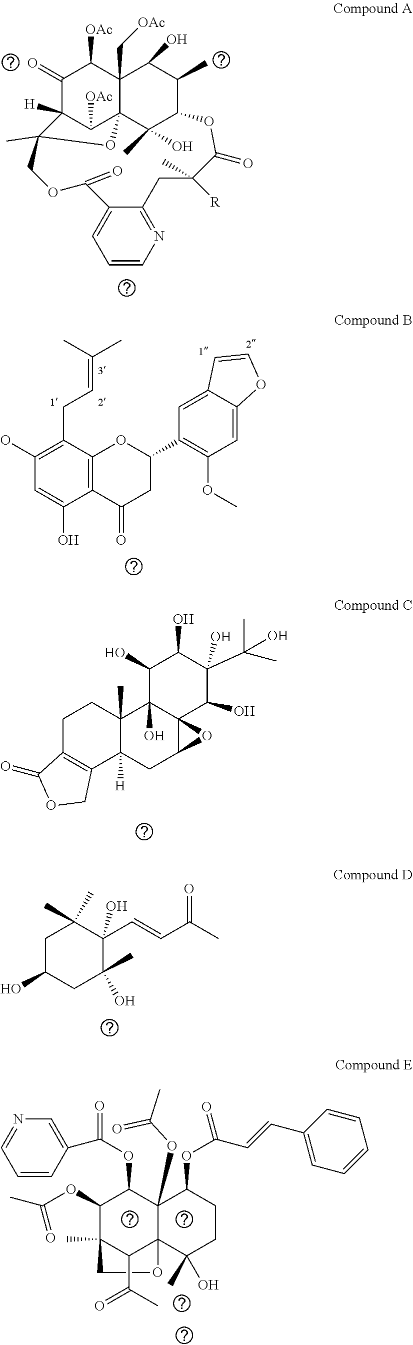

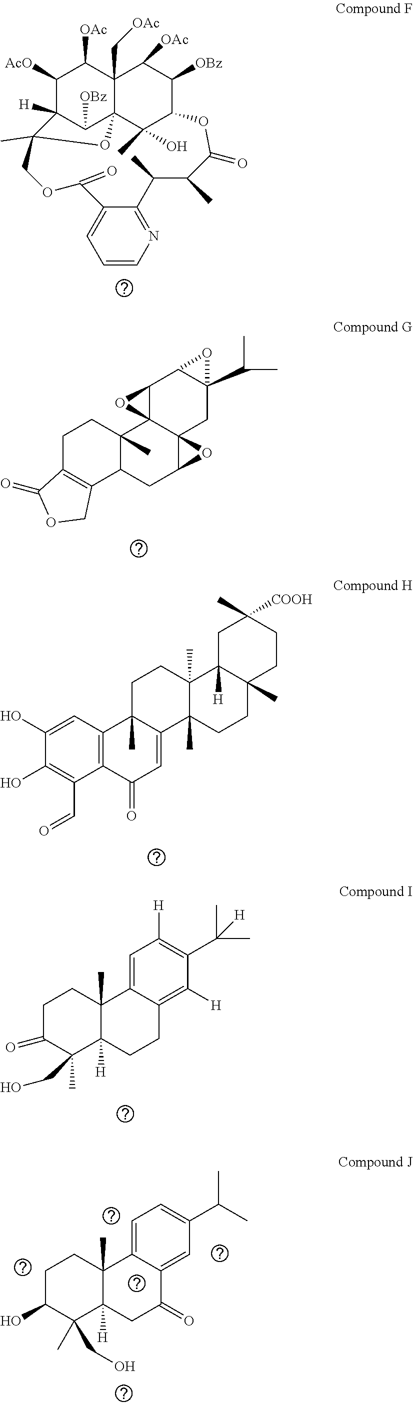

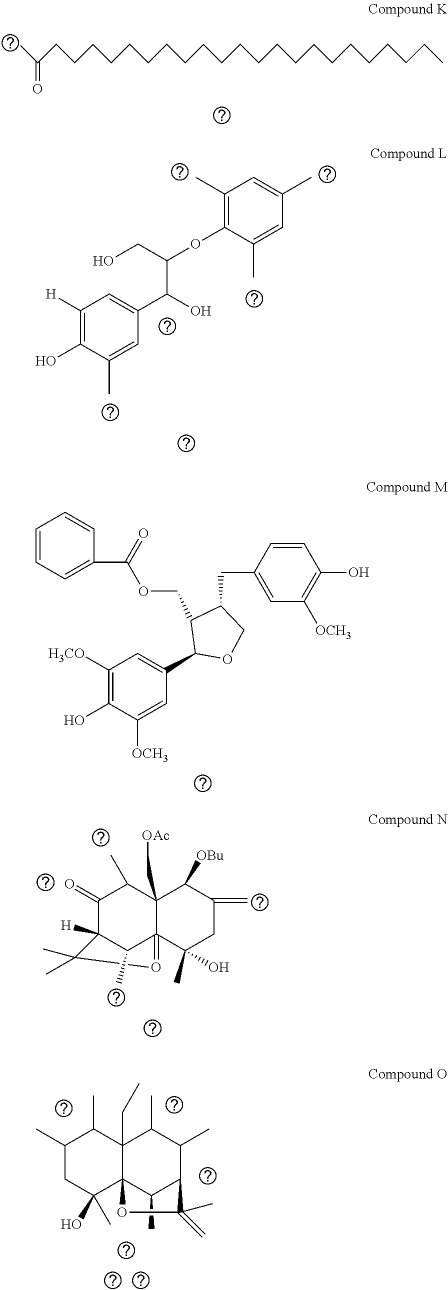

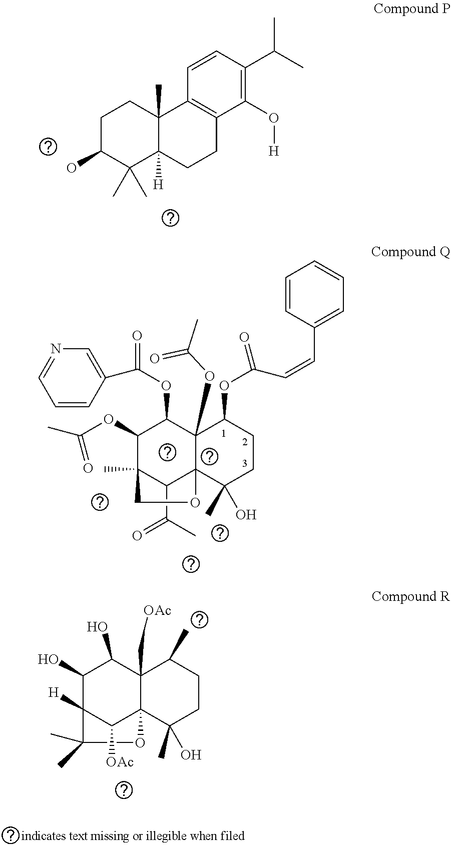

In the present disclosure, the composition is a combination of the G and one or more selected from the group consisting of the E, the H, the I, the J, and the P. In the present disclosure, when the composition is a combination of the G and one compound of the E, the H, the I, the J, and the P, the G and the one compound are at a volume ratio of 1:4 to 4:3, preferably 1:1. When the composition is a combination of the G and two compounds of the E, the H, the I, the J, and the P, the G and the two compounds are at a volume ratio of 1:1:1 to 4:3:4, preferably 1:1:1.

In the present disclosure, the G, the E, the I, and the J each have an effective concentration of 0.5 μg/ml to 7 μg/ml. The G has an effective concentration of preferably 2.5 μg/ml to 5 μg/ml, the E has an effective concentration of preferably 1 μg/ml to 2 μg/ml, the I has an effective concentration of preferably 0.9 μg/ml to 1.8 μg/ml, and the J has an effective concentration of preferably 2 μg/ml to 4 μg/ml.

In the present disclosure, the H and the P each have an effective concentration of 10 μg/ml to 45 μg/ml. The H has an effective concentration of preferably 11 μg/ml to 22 μg/ml, and the P has an effective concentration of preferably 20 μg/ml to 40 μg/ml.

The present disclosure further provides a preparation method of the Tripterygium wilfordii composition, including the following steps: subjecting compounds in a mixture of a Tripterygium wilfordii suspension cell extracting solution-derived analytical sample and a Tripterygium wilfordii suspension cell culture solution-derived analytical sample to high-performance liquid chromatography (HPLC) separation, and combining obtained separated compounds.

In the present disclosure, a preparation method of the Tripterygium wilfordii suspension cell extracting solution-derived analytical sample specifically includes: Tripterygium wilfordii suspension cells are dried, Soxhlet reflux extraction is conducted with 20 mL of ethyl acetate 3 times, three obtained extracts are combined, the solvent ethyl acetate is recovered by a rotary evaporator, an obtained residue is evaporated to dryness and dissolved in 1 ml of methanol, 500 μl of a resulting methanol solution is mixed with 3 g of a silica gel powder for sample blending, and sample loading is conducted at an amount of silica gel filler of 20 g. The sample is rinsed with 200 mL of a petroleum ether eluent, and the eluent is discarded. The sample is sequentially eluted with 200 mL of a petroleum ether: ethyl acetate eluent of different ratios (9:1, 8:2, 7:3, 6:4, 5:5, 4:6, 3:7, 2:8, 1:9, and 0:10), and 10 fractions of eluates are collected. Each eluate is subjected to solvent recovery with a rotary evaporator, each obtained residue is evaporated to dryness, and dissolved with 1 ml of chromatographically pure methanol to form a 100 μg/mL solution, and each solution is passed through a 0.22 μm microporous membrane to obtain the suspension cell extracting solution-derived analytical sample.

In the present disclosure, a preparation method of the Tripterygium wilfordii suspension cell culture solution-derived analytical sample specifically includes: separating a culture solution by vacuum filtration; mixing an obtained separated culture solution fully with an equal volume of petroleum ether, and conducting extraction three times until a petroleum ether layer is colorless; extracting an obtained treated culture solution 3 times with 1/2 volume of ethyl acetate, combining three obtained extracts, recovering the solvent ethyl acetate by a rotary evaporator, and passing through a 0.22 μm microporous membrane to obtain the Tripterygium wilfordii suspension cell culture solution-derived analytical sample.

In the present disclosure, the Tripterygium wilfordii suspension cell extracting solution-derived analytical sample and the Tripterygium wilfordii suspension cell culture solution-derived analytical sample are combined to obtain a mixture, that is, an isolated sample solution of the composition of the present disclosure.

In the present disclosure, the HPLC separation is conducted preferably by: a chromatographic column of Hypersil ODS2-C18 250 mm, a column temperature at a room temperature, an absorption wavelength of 230 nm, a sample injection volume of 20 μl, and a mobile phase of acetonitrile and ultrapure water; and elution is conducted according to Table 1:

TABLE 1
Gradient elution conditions
for HPLC separation of products

| Time | Acetonitrile | Ultrapure | Flow rate |
|---|---|---|---|
| (min) | (%) | water (%) | (mL/min) |
| 0 | 10 | 90 | 0.5 |
| 10 | 16 | 84 | 0.5 |
| 35 | 30 | 70 | 0.5 |
| 36 | 30 | 70 | 1 |
| 50 | 30 | 70 | 1 |
| 80 | 100 | 0 | 0.5 |
| 90 | 10 | 90 | 0.5 |

In the present disclosure, after the isolated sample solution of the composition is separated by the elution conditions shown in Table 1, 18 types of compounds obtained are as follows: A: 1-desacetylwilforgine; B: (2S)-5,7-dihydroxy-2′-methoxy-8-(3-methyl-2-butenyl)-4′,5′-furanone-flavanone (Tripteryol C); C: Triptergulide D; D: (3S,5R,6S,7E)-3,5,6-trihydroxy-7-megastigmen-9-one; E: 1-(E-cinnamoyloxy)-4a-hydroxy-5a,7β,11-triacetoxy-8β-nicotinoyloxy-dihydroagarofuran; F: Tripterygiumine C; G: triptolide (TP); H: Demethylzeylasteral; I: Triptobenzene R; J: (3S,4S,5R,10S)-3,19-dihydroxy-7-ox-oabieta-8,11,13-triene; K: Tricosanoic acid; L: Wilfordiol B ((7R,8S)-8-O-4′-(3′,5′-dimethoxy-1′-hydroxymethylphenyl)-guaiacylglycerol); M: (+)-(7R,8S,8'S)-9-benzoyloxy-5-methoxy-lariciresinol; N: Triptersinine O (1β,5a-difuroyloxy-4a-hydroxy-7β,11-diacetoxy-8β-nicotinoyloxy-dihydroagarofuran); O: Triptregeline J (2β-acetoxy-1a,9β-dibenzoyloxy-4β-hydroxy-6β-(3-nicotinoyloxy)-O-dihydroagarofuran); P: Triptobenzene B; Q: Triptersinine E (1β-Z-cinnamoyloxy-4a-hydroxy-5a,7β,11-triacetoxy-8β-nicotinoyloxy-dihydroagarofuran); and R: Triptersinine Z3 (5α,11-diacetoxy-1β-trans-cinnamoyl-4α,7β,8-trihydroxy-dihydroagarofuran).

In the present disclosure, preferred compounds are obtained from the 18 compounds further separated by HPLC. The HPLC separation is conducted preferably by: a chromatographic column of Hypersil ODS2-C18 250 mm, a column temperature at a room temperature, an absorption wavelength of 230 nm, a sample injection volume of 20 μl, and a mobile phase of acetonitrile and ultrapure water; and elution is conducted according to Table 2:

TABLE 2
Gradient elution conditions
for HPLC separation of products

| Time | Acetonitrile | Ultrapure | Flow rate |
|---|---|---|---|
| (min) | (%) | water (%) | (mL/min) |
| 0 | 27 | 73 | 1 |
| 4 | 48 | 52 | 1 |
| 7 | 48 | 52 | 1 |
| 17 | 95 | 5 | 1 |
| 20 | 27 | 73 | 1 |
| 30 | 27 | 73 | 1 |

In the present disclosure, after the 18 types of compounds are separated by the elution conditions shown in Table 2, the preferred compounds obtained are as follows: A: 1-desacetylwilforgine; B: (2S)-5,7-dihydroxy-2′-methoxy-8-(3-methyl-2-butenyl)-4′,5′-furanone-flavanone (Tripteryol C); C: Triptergulide D; D: (3S,5R,6S,7E)-3,5,6-trihydroxy-7-megastigmen-9-one; F: Tripterygiumine C; G: triptolide (TP); H: Demethylzeylasteral; I: Triptobenzene R; M: (+)-(7R,8S,8'S)-9-benzoyloxy-5-methoxy-lariciresinol; N: Triptersinine O (1β,5a-difuroyloxy-4a-hydroxy-7β,11-diacetoxy-8β-nicotinoyloxy-dihydroagarofuran); P: Triptobenzene B; and R: Triptersinine Z3 (5α,11-diacetoxy-1β-trans-cinnamoyl-4α,7β,8β-trihydroxy-dihydroagarofuran).

The present disclosure further provides use of the Tripterygium wilfordii composition in preparation of a drug for inhibiting a drug-resistant cancer. In the present disclosure, the cancer includes any one or more of lung cancer, gastric cancer, pancreatic cancer, breast cancer, and leukemia. The drug includes any one or more of suppository, tablet, pill, granule, film, microcapsule, drop pill, aerosol, medicinal liquor, syrup, oral liquid, injection, or injection powder.

The technical solutions provided by the present disclosure will be described in detail below with reference to examples, but the examples should not be construed as limiting the claimed scope of the present disclosure.

### Example 1 Combined Inhibitory Effect of Compounds on Cisplatin-Resistant Cells A549/DDP

1. Cell Model Establishment

The cisplatin-resistant cells of non-small cells, lung cancer cells A549 and H1299 and human large cells, lung cancer cells H460 (that is, A549/DDP, H1299/DDP, and H460/DDP), as well as normal human lung bronchial epithelial cells 16HBE were used as cultured cells. All the cells were cultured with a PRMI-1640 complete medium at 37° C. with a CO₂ concentration of 5%. Three kinds of cell models were established by culturing the above cells to a certain amount.

2. Determination of an Inhibitory Rate of the Compounds on Cell Proliferation

The three kinds of cells in a logarithmic growth phase obtained in step 1 were separately inoculated in a 96-well plate at a density of 5×10⁴ cells/mL. 90 μL of the cells were added to each well, and a complete medium as a blank control group, a DMSO negative control, a TP positive control, and experimental groups with different concentrations of compounds were set up. Three replicate wells were set up in each group, and the cells were cultured in a carbon dioxide incubator for 24 h. Drugs were added in a volume of 10 μl to each well, such that final concentrations were 10 μg/ml, 20 μg/ml, 30 μg/ml, 40 μg/ml, and 50 μg/ml. After culturing for 24 h, 48 h, and 72 h, 10 μl of an MTT solution was added to each well. After incubation for 4 h, the medium in each well was aspirated, and 100 μl of DMSO was added. The culture plate was placed in a microplate reader and shaken for 5 min to dissolve crystals. The cells were put into a microplate reader for detection, and a detection wavelength was set to OD=490 nm and a reference wavelength OD=630 nm to measure an absorbance of each well.

The formulas for relative cell viability or relative cell inhibition rate were as follows:

Relative cell viability (%)=((OD value of drug experimental group−OD value of blank control group)/(OD value of negative control group−OD value of blank control group))×100%.

Relative cell inhibition rate (%)=(1−relative cell viability)×100%.

The formula for calculating a combination index (CI) was as follows:

$$\frac{{( D )}_{1}}{{( {D}_{x} )}_{1}} + \frac{{( D )}_{2}}{{( {D}_{x} )}_{2}} + \frac{{( D )}_{3}}{{( {D}_{x} )}_{3}} + … + \frac{{( D )}_{n}}{{( {D}_{x} )}_{n}} = C I$$

In the formula, (Dₓ)₁, (Dₓ)₂, and (Dₓ)₃ represented the drug doses used alone, while (D)₁, (D)₂, and (D)₃ represented the inhibition rates contributed by the respective drugs in the combination. The drug combination index software CompuSyn was used for modeling evaluation to determine whether the two drugs had a mutual synergistic effect. When CI<1, there was a synergistic effect; when CI>1, there was an antagonistic effect; and when CI=1, there was a duplicate effect.

A calculation method of IC₅₀ was as follows: SPSS software was used for calculation: after entering the SPSS data editor, the (Variable View) was activated to define variables, and under the variable name (Name), informations were input as follows: “dose/concentration”, “inhibition rate”, “total value”. A parameter Analysis Regression Probit was selected, the “Probit Analysis” dialog box was brought up, the “Dose/Concentration” was selected into the “Covariates” column, the “Inhibition Rate” was selected into the “Response Frequency” column, and the “Total Value” was selected into the “Total Observed” column. In the “Transform” column, “Log base 10” was selected; in the “Mode 1” column, the “Probit” probability unit model was selected; and in the “Options” column, “Calculate from Data” was selected. In this way, the dose value showing each effect probability level was displayed, namely the IC₅₀ value.

3. Analysis of the Results of Compounds in Inhibiting the Proliferation of Tumor Drug-Resistant Cells

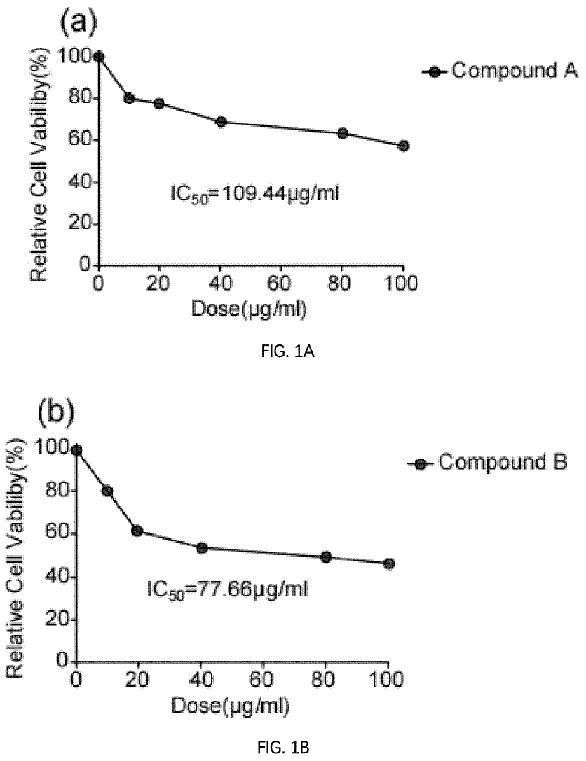

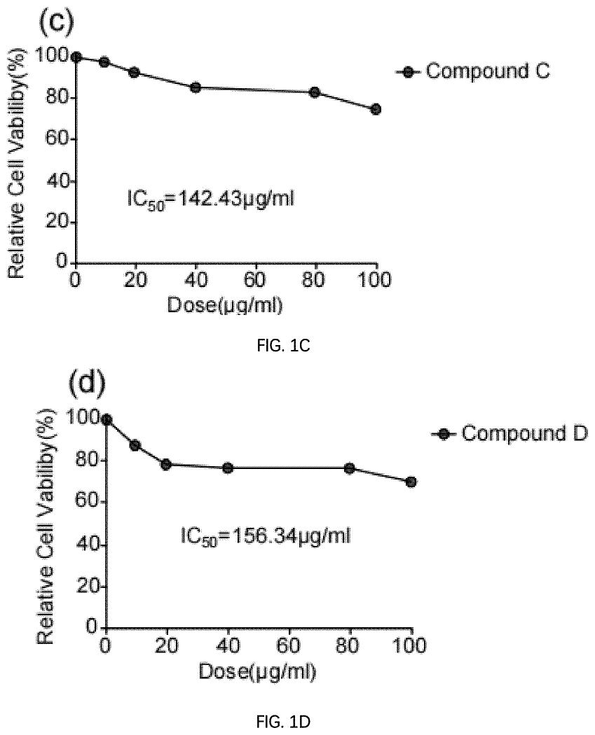

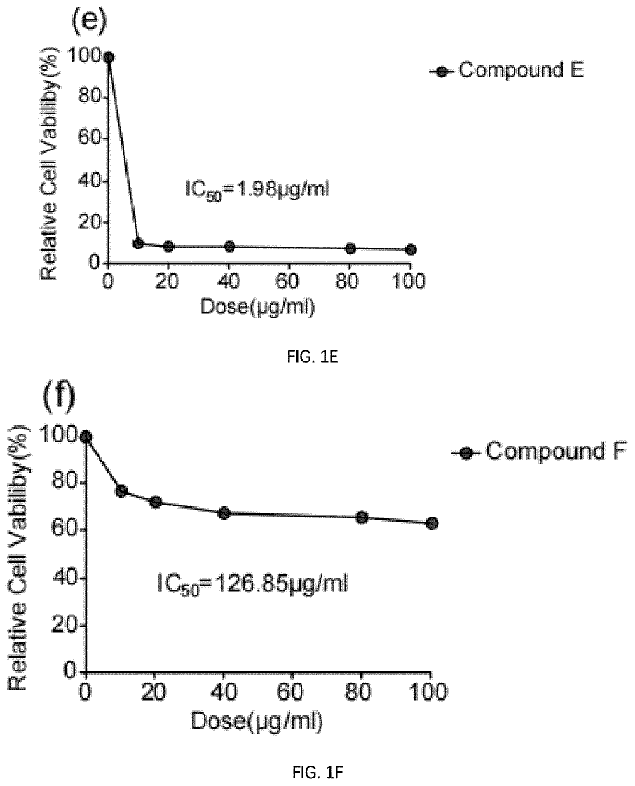

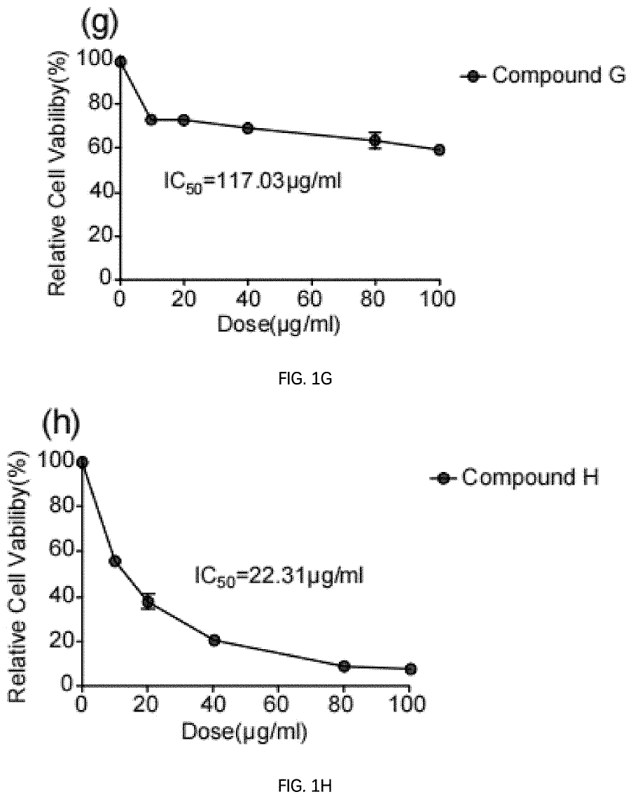

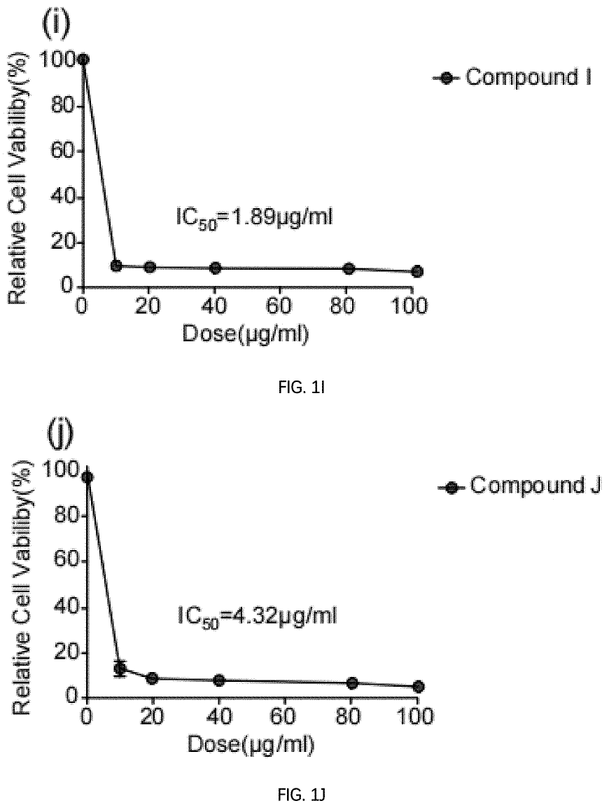

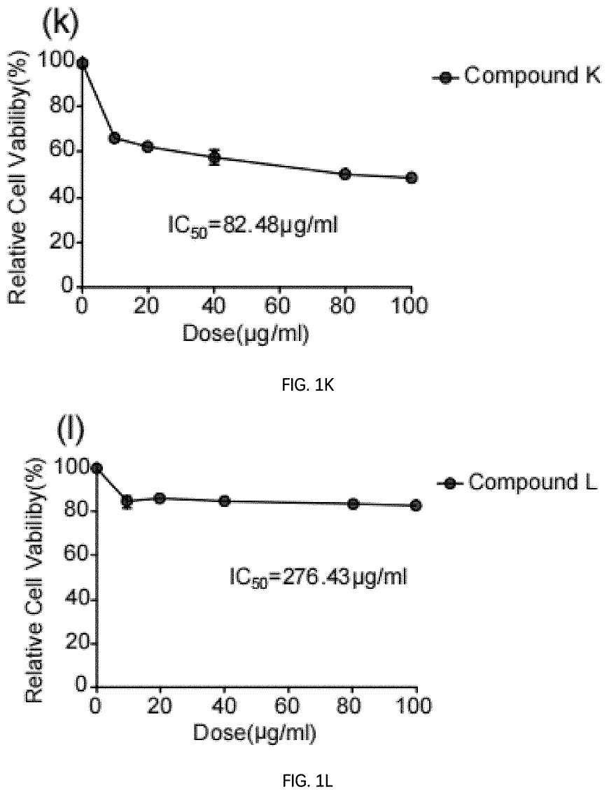

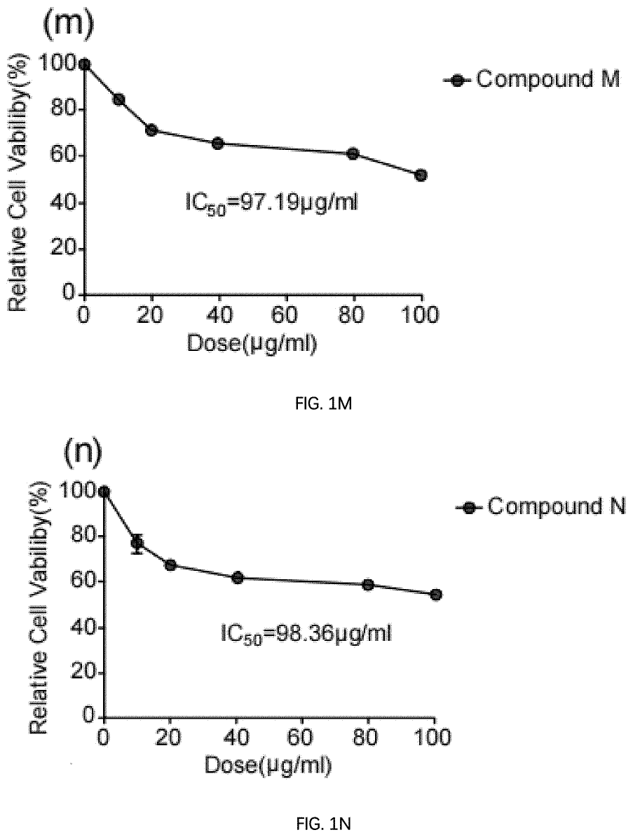

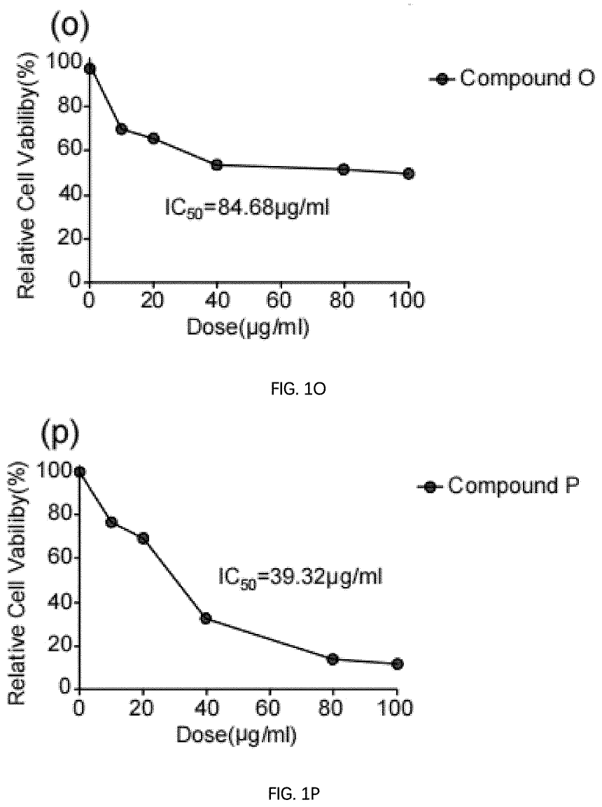

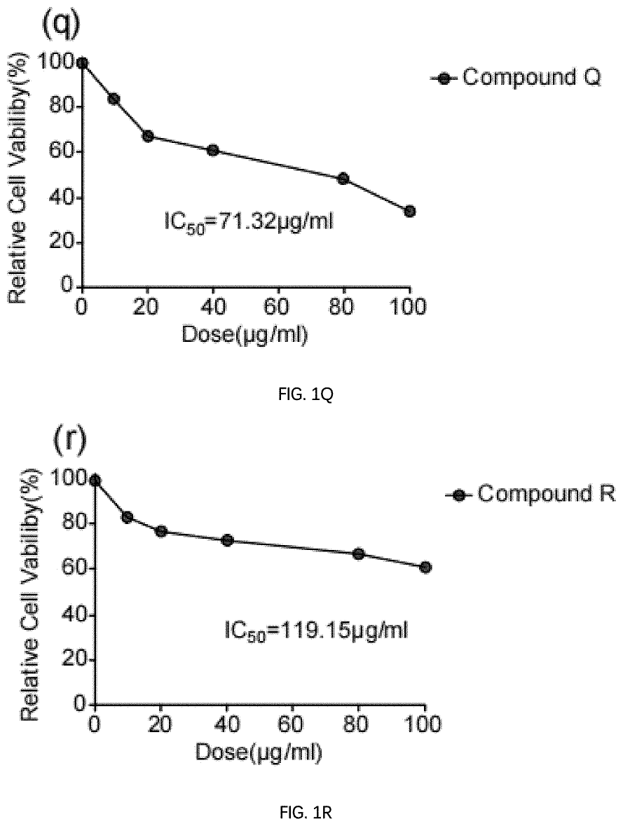

As shown in FIGS. 1A-R, the 8 types of compounds of the present disclosure exhibited different drug effects. Among them, the five compounds E, H, I, J, and P had the strongest inhibitory activity on cell proliferation. The IC₅₀ of compound E was 1.98 μg/ml, and the IC₅₀ of compound I was 1.893 μg/ml, which were comparable to the IC₅₀ value of the positive drug TP (1.97 μg/ml). However, the compounds H, J and P were relatively weaker, with IC₅₀ values of 22.31 μg/ml, 4.32 μg/ml, and 39.20 μg/ml, respectively.

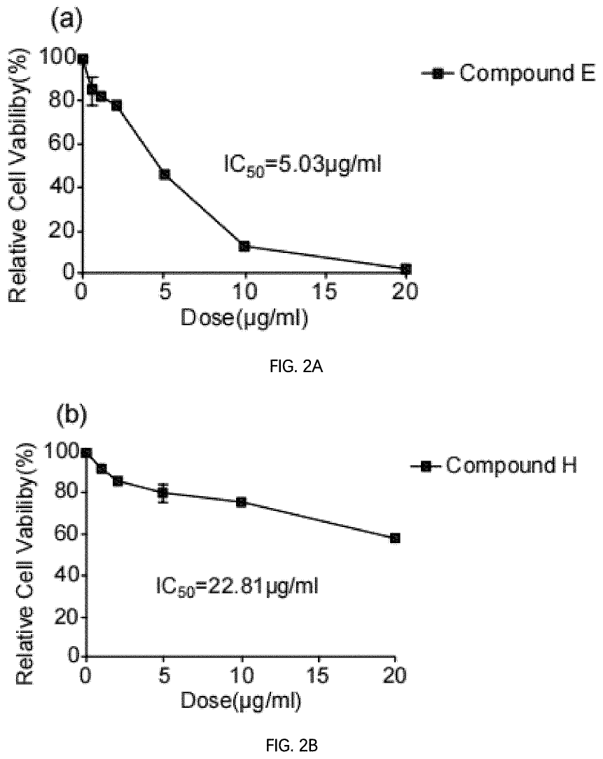

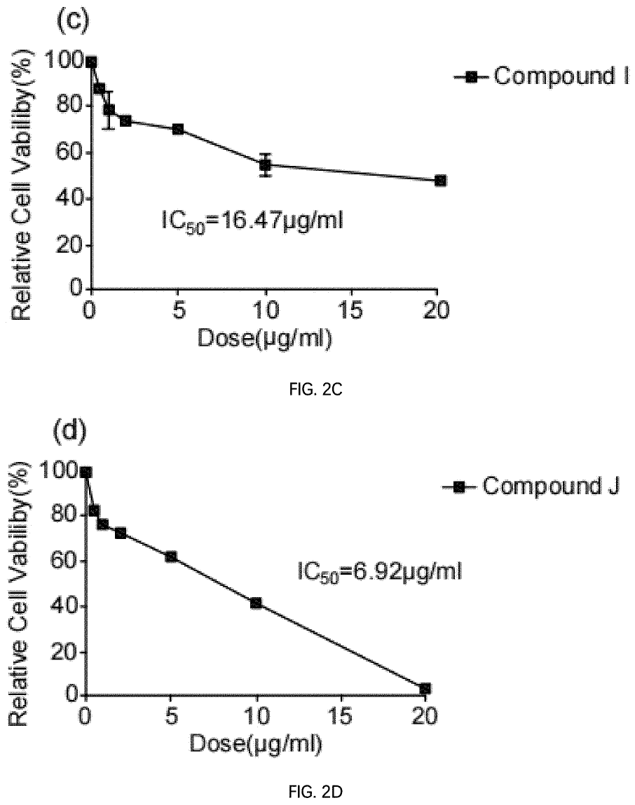

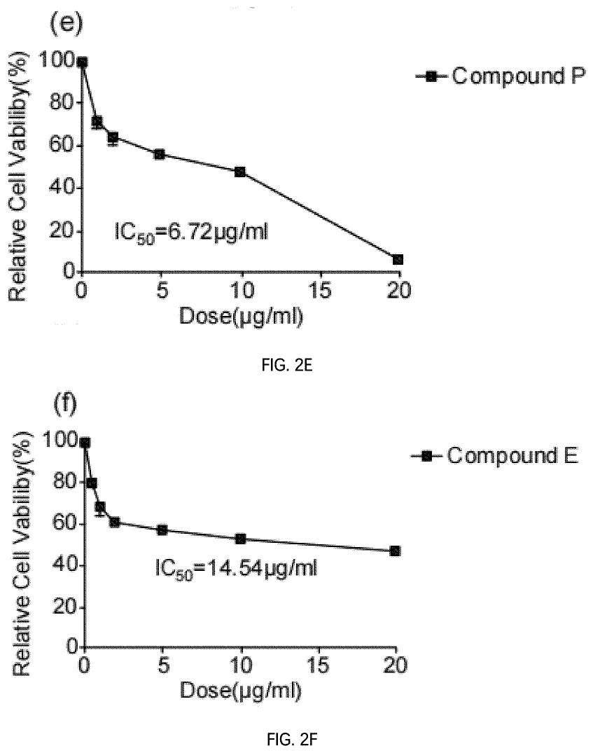

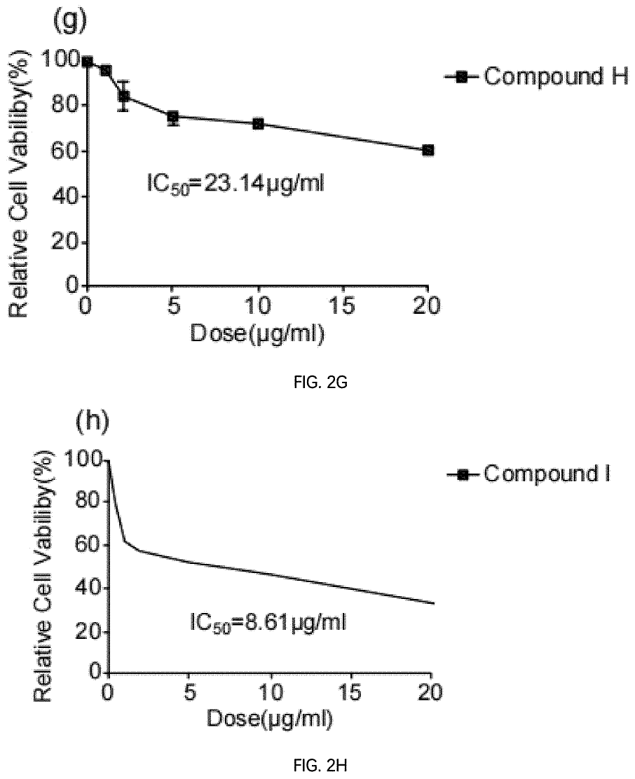

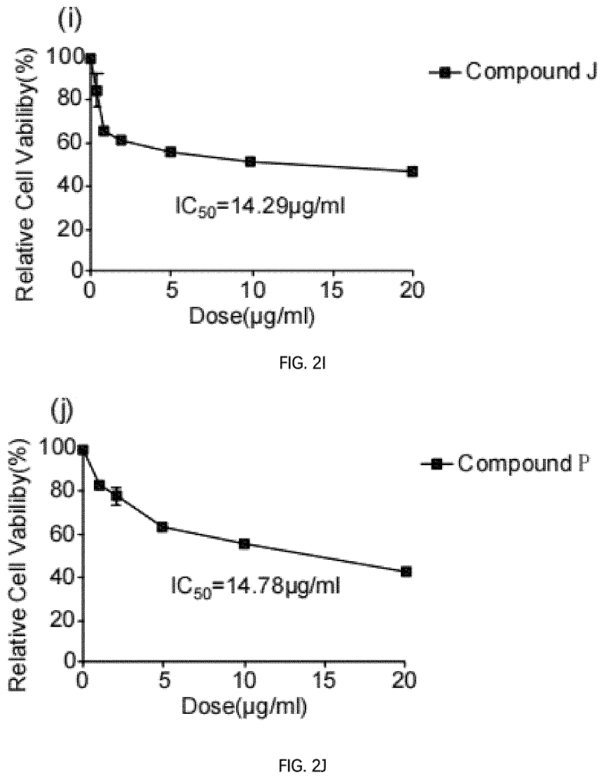

As shown in FIGS. 2A-J, the E, J, and P had significant inhibitory effects on the proliferation of A549 cells, and their IC₅₀ were 5.03 μg/ml, 6.92 μg/ml, and 6.72 μg/ml, respectively. However, the compound I was the most sensitive to H1299 cells with an IC₅₀ of 8.61 μg/ml.

From the test results in FIGS. 1A-R to FIGS. 2A-J, the five compounds, E, H, I, J, and P, were analyzed and optimized, which were confirmed to have a synergistic drug effect with the TP. The IC₅₀ value of A549DDP cells with the most sensitive drug effect was used as a synergistic dose, TP was 7.83 μg/ml, and the IC₅₀ of the five active compounds E, H, I, J, and P were 2 μg/ml, 22 μg/ml, 1.8 μg/ml, 4 μg/ml, and 40 μg/ml, respectively.

4. Analysis of Combined Drug Effects of the 5 Compounds on A549/DDP Cells

The IC₅₀ value of TP at half of the dose 7.83 μg/ml, that is, 4 μg/ml was used as its drug concentration. The IC₅₀ of the five active compounds E, H, I, J, and P were halved, and the selected concentrations were 1 μg/ml, 11 μg/ml, 0.9 μg/ml, 2 μg/ml, and 20 μg/ml, respectively. When drugs were used in combination, the TP was compatible with any one or any two of the compounds E, H, I, J, and P separately in an equal volume ratio. The analysis results were shown in FIGS. 3A-E. For A549/DDP, compared with the control group, all five compounds had a significant growth inhibitory activity. The compound I had a better inhibitory effect on cells than that of the TP, while the other four compounds had a similar antitumor effect to that of the TP. Compared with the single TP group, the proliferation inhibitory effect of the two drugs combined was significantly improved. Compared with the single drug group, the two drugs combined had a cell inhibition rate increased by 2.01, 1.54, 1.81, 1.58, and 1.34 times, showing a significant synergistic effect with TP-combined drugs.

Further, the drug combination index software CompuSyn was used for modeling evaluation to determine whether the two drugs had a mutual synergistic effect. When CI<1, there was a synergistic effect; when CI>1, there was an antagonistic effect; and when CI=1, there was a duplicate effect.

For A549/DDP cells, the results were shown in Table 3 and FIGS. 4A-E. The combination index of the five compounds ranged from 0.7206 to 0.9982. According to the drug effect of their combined anti-tumor activity, the strength effects were: Compound H>Compound I>Compound J>Compound E>Compound P. Herein, after the compound J was used in combination with TP, there was the most significant cell inhibition effect, and its inhibition rate was 1.32 times higher that of TP alone. According to the principle that the smaller the CI value, the stronger the synergistic effect, it was found that when the dose of compound H was 22 μg/ml and the dose of compound J was 2 μg/ml, the synergistic effect was the best when these two compounds were combined with 5 μg/ml TP.

TABLE 3
Combination index (CI) of compounds and TP acting
alone and in combination on A549/DDP cells (n = 3)

| | | TP (μg/ml) | |
|---|---|---|---|
| Compound (μg/ml) | | 2.5 | 5 |
| Compound E | 1 | 0.8986 | 0.8688 |
| | 2 | 1.1269 | 1.1213 |
| Compound H | 11 | 0.8749 | 1.1462 |
| | 22 | 0.7225 | 0.7206 |
| Compound I | 0.9 | 1.3864 | 0.8316 |
| | 1.8 | 0.8583 | 0.8288 |
| Compound J | 2 | 0.9333 | 0.6952 |
| | 4 | 0.9025 | 0.9982 |
| Compound P | 20 | 1.0764 | 1.1918 |
| | 40 | 1.2660 | 1.4985 |

### Example 2 Combined Inhibitory Effect of Five Compounds on Cisplatin-Resistant Cells H1229/DDP

The experimental design, the five compounds, and a method for determining the inhibitory effect on cisplatin-resistant cells H1229/DDP were the same as those in Example 1.

As shown in FIGS. 5A-E, compared with the control group, all five compounds had significant anti-proliferation activity on H1299/DDP cells. The inhibitory activity of the compound P was stronger than that of TP, with IC₅₀ of 12.80 μg/ml. It was found that after the combination of the two drugs, the proliferation inhibitory effect was significantly improved. Compared with the single drug group, the combination of the two drugs increased the cell inhibition rate by 2.08, 3.24, 3.19, 2.36, and 2.06 times, showing a significant synergistic effect with the TP-combined drug.

For H1299/DDP cells, the results were shown in Table 4 and FIGS. 6A-E. When the TP concentration dose were 2.5 μg/ml, the drug combination index of the five compounds from large to small was as follows: Compound P>Compound E>Compound I>Compound J>Compound H, corresponding to IC₅₀ concentrations doses of 12.0 μg/ml, 0.84 μg/ml, 0.9 μg/ml, 1.84 μg/ml, and 8.58 μg/ml. When the dose of TP was 5 μg/ml, except the compound I whose dose was 1.8 μg/ml, and the drug combination index was 1.1503>1, the other compounds showed a synergistic effect. The effect ranking was: Compound E>Compound J>Compound H>Compound P>Compound I, corresponding to IC₅₀ concentrations doses of 1.0 μg/ml, 0.9 μg/ml, 4.0 μg/ml, 11.0 μg/ml, 20.0 μg/ml. After the compound E and the compound H were used in combination with the TP, the inhibition rates reached 91.13% and 83.50%, respectively. Compared with TP alone, the drug effects of these two drugs increased by 2.15 and 2.12 times, respectively, showing a significant drug synergistic effect.

A smaller CI value indicated a desirable synergistic effect. From the above experimental results, in the H1299/DDP cell model, the TP at a concentration of 5 μg/ml was combined with the compound E at a concentration of 1 μg/ml, the compound H at concentration of 11 μg/ml, and the compound at concentration of 4 μg/ml. It was found that the corresponding CI values were all less than 0.6, indicating that the three drugs showed a desirable synergistic effect.

TABLE 4
Combination index (CI) of compounds and TP acting alone
and in combination on H1299/DDP cells after 48 h (n = 3)

| | | TP (μg/ml) | |
|---|---|---|---|
| Compound (μg/ml) | | 2.5 | 5 |
| Compound E | 1 | 0.7473 | 0.5193 |
| | 2 | 1.1646 | 0.8025 |
| Compound H | 11 | 0.8151 | 0.5827 |
| | 22 | 0.9651 | 0.8689 |
| Compound I | 0.9 | 0.7373 | 0.8353 |
| | 1.8 | 1.1259 | 1.1503 |
| Compound J | 2 | 0.6741 | 0.6735 |
| | 4 | 0.9855 | 0.5884 |
| Compound P | 20 | 1.7041 | 0.6521 |
| | 40 | 1.1079 | 0.9326 |

### Example 3 Combined Inhibitory Effect of Five Compounds on Cisplatin-Resistant Cells H460/DDP

The experimental design, the five compounds, and a method for determining the inhibitory effect on cisplatin-resistant cells H460/DDP were the same as those in Example 1.

As shown in FIGS. 7A-E, compared with the control group, all five compounds had a significant inhibitory activity on H460/DDP cells. Among them, compounds E and J had a stronger inhibitory activity on cells than that of TP, with IC₅₀ of 0.58 μg/ml and 0.61 μg/ml, respectively. Moreover, it was found that the proliferation inhibitory effect of the two drugs was significantly improved after the combined action of the two drugs. Compared with the single drug group, the cell inhibition rate increased by 1.23, 1.72, 1.45, 1.33, and 1.53 times, showing a synergistic effect with the TP-combined drug.

For the H460/DDP cells, the results were shown in FIGS. 8A-E and Table 5. According to the drug combination index, when the TP dose was 2.5 μg/ml, the drug combination index of the five compounds in descending order was: Compound I>Compound E>Compound H>Compound J>Compound P. After combined with TP, the inhibition rates were 91.82%, 94.05%, 99.56%, and 86.80%, which were 1.52, 1.56, 1.65, and 1.44 times higher than that of TP alone, respectively. This showed that the synergistic effect was remarkable, and the antitumor activity was obviously improved.

After the compound J and the compound P were administered in combination with the TP, the CI values acting on H460/DDP cells were both less than 0.35. Through modeling, it was found that the CI value of compound P combined with TP reached 0.1961 at a dose of 40 μg/ml, indicating that the synergistic effect of the compound P combined with TP was more prominent at this concentration dose.

TABLE 5
Combination index (CI) of compounds and TP acting alone
and in combination on H460/DDP cells after 48 h (n = 3)

| | | TP (μg/ml) | |
|---|---|---|---|
| Compound (μg/ml) | | 2.5 | 5 |
| Compound E | 1 | 1.4669 | 1.1134 |
| | 2 | 0.6604 | 0.5008 |
| Compound H | 11 | 0.7051 | 0.6318 |
| | 22 | 0.5569 | 0.4270 |
| Compound I | 0.9 | 1.1241 | 0.9327 |
| | 1.8 | 1.2499 | 0.7619 |
| Compound J | 2 | 0.5384 | 0.6519 |
| | 4 | 0.3260 | 0.3064 |
| Compound P | 20 | 0.2435 | 0.3777 |
| | 40 | 0.1961 | 0.2976 |

### Example 4 Attenuation Effect of Various Compositions on Normal 16HBE Cells

The experimental design, the five compounds, and a method for determining the inhibitory effect on cisplatin-resistant cells H460/DDP were the same as those in Example 1.

As shown in FIGS. 9A-E, compared with the control group, after TP was combined with the five compounds, the relative cell viabilities were 75.09%, 79.44%, 78.83%, 78.86%, and 73.69%, respectively. Compared with TP alone, the cytotoxicity was reduced by 1.16, 1.24, 1.21, 1.22, and 1.14 times, respectively. This reduced the toxicity of TP alone to normal cells to a certain extent, and improved the safety. The combined drug also showed a certain compatibility attenuation effect.

Based on the measurement and analysis results of Examples 1 to 4, it was seen that for A549/DDP cells, the compound H at 22 μg/ml, the compound J at 2 μg/ml, and the TP at 5 μg/ml combined at a volume ratio of 1:1:1 formed an optimal combination. For H1299/DDP cells, 5 μg/ml of the TP was mixed with two of the compound E at 1 μg/ml, the compound H at 11 μg/ml, and the compound J at 4 μg/ml at a volume ratio of 1:1:1 to form an optimal combination. For H460/DDP cells, the compound P at 40 μg/ml and the TP at 5 μg/ml formed an optimal combination at a volume ratio of 1:1. Finally, any one of TP/H/J, TP/E/H, and TP/P was selected as a compatible composition.

### Example 5 Inhibitory Effects of Three Compatible Compositions on Drug-Resistant Cells of Different Tumors

The cisplatin-resistant strains of SGC7901/DDP MXC336 human gastric cancer, the fluorouracil-resistant strains of HL-60/FU human leukemia, the gemcitabine-resistant strains of PANC-1/GEM human pancreatic cancer cells, and the oxaliplatin-resistant cell line of MCF7/L MXC804 human breast cancer were used as cell models. Three kinds of compositions were selected as drugs, among which TP/H/J and TP/E/H both had a drug dose ratio of 1:1:1 to 4:4:4 separately, and TP/P had a drug dose ratio of 1:1 to 4:4. The experimental method and calculation method were conducted according to those in Example 1, and calculated IC₅₀ values of each group were shown in Table 6. Through comparison, it was seen that each composition had a highly desirable inhibitory effect on the four drug-resistant cells. The compositions were more sensitive to the cisplatin-resistant cells of human gastric cancer, with the strongest inhibitory effect of the drug. The optimal volume ratios of TP/H/J and TP/E/H were both 1:1:1, and the optimal volume ratio of TP/P was 1:1. The compatibility of the above limited three compositions and dose ratios had the strongest and most effective drug effect.

TABLE 6
Inhibitory effects of three compositions on different drug-resistant cells

| | Drug | IC₅₀ (μg/ml) | | | |
|---|---|---|---|---|---|
| | compatibility | SGC7901/ | HL-60/ | PANC-1/ | MCF7/ |
| Composition | volume ratio | DDP | FU | GEM | LMXC804 |
| TP/H/J | 1:1:1 | 0.6834 | 2.4673 | 1.2204 | 0.9014 |
| | 1:2:3 | 1.2406 | 4.1301 | 2.1907 | 1.4201 |
| | 4:3:4 | 1.5092 | 5.8362 | 2.7619 | 1.6309 |
| TP/E/H | 1:1:1 | 0.1801 | 1.0203 | 0.3732 | 0.1939 |
| | 1:3:2 | 0.4362 | 2.1106 | 0.8131 | 0.8617 |
| | 4:3:4 | 1.0231 | 3.0017 | 1.0726 | 1.0806 |
| TP/P | 1:1 | 0.3103 | 2.5301 | 0.5011 | 0.4127 |
| | 1:3 | 0.5432 | 2.8912 | 1.0105 | 1.8321 |
| | 4:3 | 1.9021 | 4.3742 | 1.6341 | 2.2176 |

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A Tripterygium wilfordii composition for inhibiting a drug-resistant cancer, comprising any two or more of the following compounds:
A: 1-desacetylwilforgine;
B: (2S)-5,7-dihydroxy-2′-methoxy-8-(3-methyl-2-butenyl)-4′,5′-furanone-flavanone (Tripteryol C);
C: Triptergulide D;
D: (3S,5R,6S,7E)-3,5,6-trihydroxy-7-megastigmen-9-one;
E: 1β-(E-cinnamoyloxy)-4a-hydroxy-5a,7β,11-triacetoxy-8β-nicotinoyloxy-dihydroagarofuran;
F: Tripterygiumine C;
G: triptolide (TP);
H: Demethylzeylasteral;
I: Triptobenzene R;
J: (3S,4S,5R,10S)-3,19-dihydroxy-7-ox-oabieta-8,11,13-triene;
K: Tricosanoic acid;
L: Wilfordiol B;
M: (+)-(7R,8S,8'S)-9-benzoyloxy-5-methoxy-lariciresinol;
N: Triptersinine O;
O: Triptregeline J;
P: Triptobenzene B;
Q: Triptersinine E; and
R: Triptersinine Z3.

2. The Tripterygium wilfordii composition according to claim 1, wherein the composition is a combination of the G and one or more selected from the group consisting of the E, the H, the I, the J, and the P.

3. The Tripterygium wilfordii composition according to claim 2, wherein when the composition is a combination of the G and one compound of the E, the H, the I, the J, and the P, the G and the one compound are at a volume ratio of 1:4 to 4:3.

4. The Tripterygium wilfordii composition according to claim 2, wherein when the composition is a combination of the G and two compounds of the E, the H, the I, the J, and the P, the G and the two compounds are at a volume ratio of 1:1:1 to 4:3:4.

5. The Tripterygium wilfordii composition according to claim 1, wherein the G, the E, the I, and the J each have an effective concentration of 0.5 μg/ml to 7 μg/ml.

6. The Tripterygium wilfordii composition according to claim 2, wherein the G, the E, the I, and the J each have an effective concentration of 0.5 μg/ml to 7 μg/ml.

7. The Tripterygium wilfordii composition according to claim 3, wherein the G, the E, the I, and the J each have an effective concentration of 0.5 μg/ml to 7 μg/ml.

8. The Tripterygium wilfordii composition according to claim 4, wherein the G, the E, the I, and the J each have an effective concentration of 0.5 μg/ml to 7 μg/ml.

9. The Tripterygium wilfordii composition according to claim 1, wherein the H and the P each have an effective concentration of 10 μg/ml to 45 μg/ml.

10. The Tripterygium wilfordii composition according to claim 2, wherein the H and the P each have an effective concentration of 10 μg/ml to 45 μg/ml.

11. The Tripterygium wilfordii composition according to claim 3, wherein the H and the P each have an effective concentration of 10 μg/ml to 45 μg/ml.

12. The Tripterygium wilfordii composition according to claim 4, wherein the H and the P each have an effective concentration of 10 μg/ml to 45 μg/ml.

13. A preparation method of the Tripterygium wilfordii composition according to claim 1, comprising the following steps: subjecting compounds in a mixture of a Tripterygium wilfordii suspension cell extracting solution-derived analytical sample and a Tripterygium wilfordii suspension cell culture solution-derived analytical sample to high-performance liquid chromatography (HPLC) separation, and combining obtained separated compounds.

14. The preparation method according to claim 13, wherein the composition is a combination of the G and one or more selected from the group consisting of the E, the H, the I, the J, and the P.

15. The preparation method according to claim 14, wherein when the composition is a combination of the G and one compound of the E, the H, the I, the J, and the P, the G and the one compound are at a volume ratio of 1:4 to 4:3.

16. The preparation method according to claim 14, wherein when the composition is a combination of the G and two compounds of the E, the H, the I, the J, and the P, the G and the two compounds are at a volume ratio of 1:1:1 to 4:3:4.

17. The preparation method according to claim 13, wherein the G, the E, the I, and the J each have an effective concentration of 0.5 μg/ml to 7 μg/ml.

18. The preparation method according to claim 13, wherein the HPLC separation is conducted at the following conditions: a chromatographic column of Hypersil ODS2-C18 250 mm, a column temperature of a room temperature, absorption wavelengths of 210 nm, 219 nm, and 230 nm, a sample injection volume of 20 μl, a mobile phase of acetonitrile and ultrapure water, and elution conditions as follows:

| Time | Acetonitrile | Ultrapure | Flow rate |
|---|---|---|---|
| (min) | (%) | water (%) | (mL/min) |
| 0 | 10 | 90 | 0.5 |
| 10 | 16 | 84 | 0.5 |
| 35 | 30 | 70 | 0.5 |
| 36 | 30 | 70 | 1 |
| 50 | 30 | 70 | 1 |
| 80 | 100 | 0 | 0.5 |
| 90 | 10 | 90 | 0.5 |

19. A method of the composition according to claim 1 in preparation of a drug for inhibiting a drug-resistant cancer.

20. The method according to claim 19, wherein the cancer comprises any one or more of lung cancer, gastric cancer, pancreatic cancer, breast cancer, and leukemia.
